(19) United States
(12) Patent Application Publication
(10) Pub. No.: US 2025/0312011 A1
(43) Pub. Date: Oct. 9, 2025

(54) POINT OF CARE ULTRASOUND INTERFACE

(71) Applicant: BFLY Operations, Inc, Burlington, MA (US)

(72) Inventors: Patrick Maher, Brooklyn, NY (US); Armend Cobovic, Burlington, MA (US); Katelyn Offerdahl, Burlington, MA (US); Francois Vignon, Andover, MA (US); Joseph Cohen, Burlington, MA (US); Abraham Neben, Guilford, CT (US); Karl Thiele, St. Petersburg, FL (US)

(73) Assignee: BFLY Operations, Inc, Burlington, MA (US)

(21) Appl. No.: 18/873,006

(22) PCT Filed: Jun. 9, 2023
(86) PCT No.: PCT/US2023/024946

§ 371 Date: Dec. 9, 2024

Related U.S. Application Data
(60) Provisional application No. 63/350,772, filed on Jun. 9, 2022.

(51) Int. Cl. A61B 8/00; A61B 8/08; G06N 3/02; G06T 7/00

(52) U.S. Cl. CPC A61B 8/465; A61B 8/08; A61B 8/4483; A61B 8/543; G06N 3/02; G06T 7/0002; A61B 2560/0487; A61B 2576/02; G06T 2200/24; G06T 2200/28

(57) ABSTRACT

A processing device, that communicates with an ultrasound device, includes: a display screen; a memory that stores presets, where each preset includes one or more modes used to control the ultrasound device and one or more tools to analyze ultrasound data from the ultrasound device; and a processor coupled to the memory. The processor is configured to: operate the ultrasound device using a first preset; generate ultrasound images using ultrasound data from the ultrasound device, where the ultrasound images include a first portion of the ultrasound images that are imaging frames acquired with the first preset and a second portion of the ultrasound images that are search frames acquired with a search preset; display the imaging frames of the first portion on the display screen; identify an anatomical feature in the search frames using a deep learning model; select a target preset based on the identified anatomical feature.

### CROSS REFERENCE TO RELATED APPLICATIONS

This application claims the benefit of priority under 35 U.S.C. § 119(e) to International Application PCT/2023/024946 filed Jun. 9, 2023, which claims the benefit of priority to U.S. Provisional Patent Application Ser. No. 63/350,772, filed on Jun. 9, 2022, which is hereby incorporated by reference herein in their entirety.

### BACKGROUND

Medical imaging may be used in performing diagnostic or therapeutic procedures. For example, ultrasound imaging uses ultrasonic waves with frequencies that are higher than those audible to humans to non-invasively visualize internal organs or soft tissue. When a probe transmits ultrasonic waves into a subject, different amplitude reflections are reflected back towards the probe from different tissue interfaces. The ultrasound image generated based on analysis of the reflections may be improved by controlling scanning and analysis parameters of the ultrasound system. For example, the field of view, ultrasound frequency ranges, frame rate, image analysis algorithms, and/or artefact compensation may be varied based on the expected anatomy. To achieve optimal ultrasound images, it is important for the parameters to be optimized based on the anatomy and/or examination type. However, point of care ultrasound (POCUS) users are often inexperienced and working under pressure. A successful user interface guides the POCUS user to the appropriate parameter settings while minimizing errors and the need for extraneous interactions with the ultrasound device.

### SUMMARY

In general, one or more embodiments of the invention relate to a processing device that communicates with an ultrasound device. The processing device includes: a display screen; a memory that stores presets, where each preset includes one or more modes used to control the ultrasound device and one or more tools to analyze ultrasound data from the ultrasound device; and a processor coupled to the memory. The processor is configured to: operate the ultrasound device using a first preset; generate ultrasound images using ultrasound data from the ultrasound device, where the ultrasound images include a first portion of the ultrasound images that are imaging frames acquired with the first preset and a second portion of the ultrasound images that are search frames acquired with a search preset; display the imaging frames of the first portion on the display screen; identify an anatomical feature in the search frames using a deep learning model; select a target preset based on the identified anatomical feature; and modify a user interface of the processing device based on the target preset. The search frames are time-interleaved with the imaging frames.

In general, one or more embodiments of the invention relate to a method of operating a processing device that communicates with an ultrasound device. The method includes: operating the ultrasound device using a first preset of a plurality of presets stored in a memory of the processing device, where each preset includes one or more modes used to control the ultrasound device and one or more tools to analyze ultrasound data from the ultrasound device; generating ultrasound images using ultrasound data from the ultrasound device, where the ultrasound images include a first portion of the ultrasound images that are imaging frames acquired with the first preset and a second portion of the ultrasound images that are search frames acquired with a search preset; displaying the imaging frames on a display screen of the processing device; identifying an anatomical feature in the search frames using a deep learning model; selecting, in response to identifying the anatomical feature, a target preset based on the identified anatomical feature; and modifying a user interface of the processing device based on the target preset. The search frames are time-interleaved with the imaging frames.

In general, one or more embodiments of the invention relate to a non-transitory computer readable medium (CRM) that stores computer readable program code for operating a processing device that communicates with an ultrasound device. The computer readable program code causes the processing device to: operate the ultrasound device using a first preset of a plurality of presets stored in a memory of the processing device, where each preset includes one or more modes used to control the ultrasound device and one or more tools to analyze ultrasound data from the ultrasound device; generate ultrasound images using ultrasound data from the ultrasound device, where the ultrasound images include a first portion of the ultrasound images that are imaging frames acquired with the first preset and a second portion of the ultrasound images that are search frames acquired with a search preset; display the imaging frames on a display screen of the processing device; identify an anatomical feature in the search frames using a deep learning model; select, in response to identifying the anatomical feature, a target preset based on the identified anatomical feature; and modify a user interface of the processing device based on the target preset. The search frames are time-interleaved with the imaging frames.

Other aspects of the invention will be apparent from the following description and the appended claims.

### BRIEF DESCRIPTION OF DRAWINGS

FIG. 1A shows an ultrasound system in accordance with one or more embodiments.

FIG. 1B shows a handheld ultrasound probe in accordance with one or more embodiments.

FIG. 1C shows a wearable ultrasound patch in accordance with one or more embodiments.

FIG. 1D shows a schematic of an ultrasound device in accordance with one or more embodiments.

FIG. 2 shows a schematic of an ultrasound system in accordance with one or more embodiments.

FIG. 3A shows a phase array of an ultrasound device in accordance with one or more embodiments.

FIGS. 3B-3E show an example of an operation of a phased array in accordance with one or more embodiments.

FIGS. 4A-4B show examples of ultrasound images acquired with different presets according to one or more embodiments.

FIG. 5 shows an example of a user interface of the processing device in accordance with one or more embodiments.

FIGS. 6A-6B show schematics of frame acquisition techniques with a search frame in accordance with one or more embodiments.

FIGS. 7A-7B show schematics of frame acquisition techniques with a line search frame in accordance with one or more embodiments.

FIG. 8 shows a schematic of a deep learning model in accordance with one or more embodiments.

FIGS. 9A-9E show examples of a processing device switching between presets in accordance with one or more embodiments.

FIG. 10 shows a flowchart of a method in accordance with one or more embodiments.

FIG. 11 shows a schematic of a computing system in accordance with one or more embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Conventional ultrasound systems are large, complex, and expensive systems that are typically only purchased by large medical facilities with significant financial resources. Recently, less expensive, and less complex ultrasound imaging devices have been introduced. Such devices may include ultrasonic transducers monolithically integrated onto a single semiconductor die to form a monolithic ultrasound device. The reduced cost and increased portability of these new ultrasound devices may make them significantly more accessible to the general public than conventional ultrasound devices. Although the reduced cost and increased portability of some ultrasound imaging devices makes them more accessible to the general populace, people who could make use of such devices have little to no training for how to use them. Ultrasound examinations often include the acquisition of ultrasound images that contain a view of a particular anatomical feature (e.g., an organ) of a subject. Acquisition of these ultrasound images typically requires considerable skill.

In general, embodiments of the disclosure provide an apparatus, a method, and a non-transitory computer readable medium (CRM) for a point of care ultrasound (POCUS) interface that aids in acquiring ultrasound images. Embodiments of the disclosure provide a method for automatically determining and implementing an appropriate set of scanning parameters to obtain an ultrasound image of a specified anatomy without user intervention. Utilizing this approach, even an inexperienced POCUS user may rapidly and efficiently be able to acquire ultrasound images.

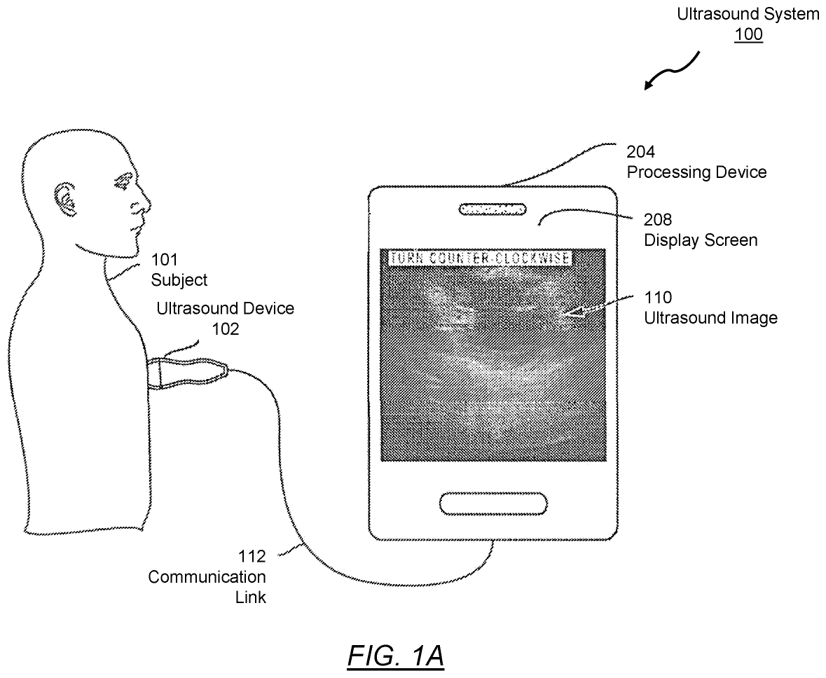

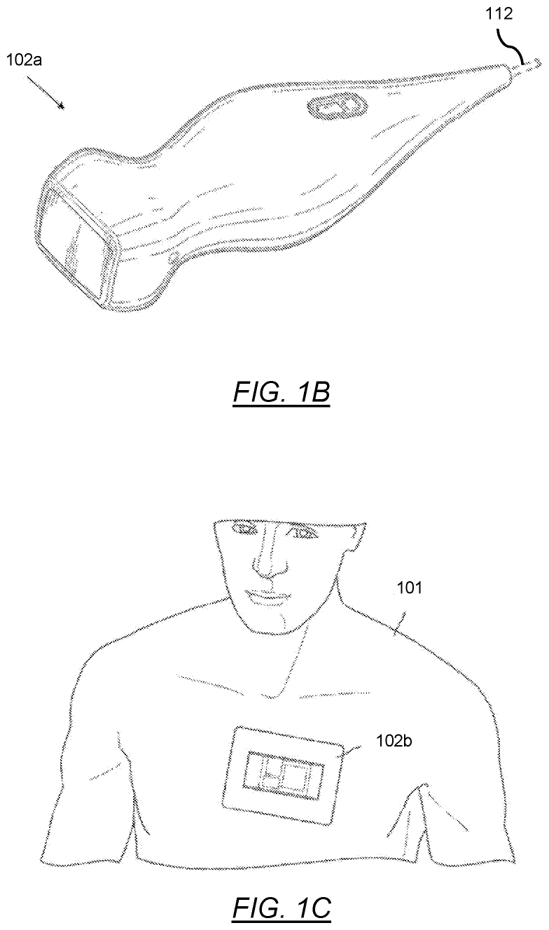

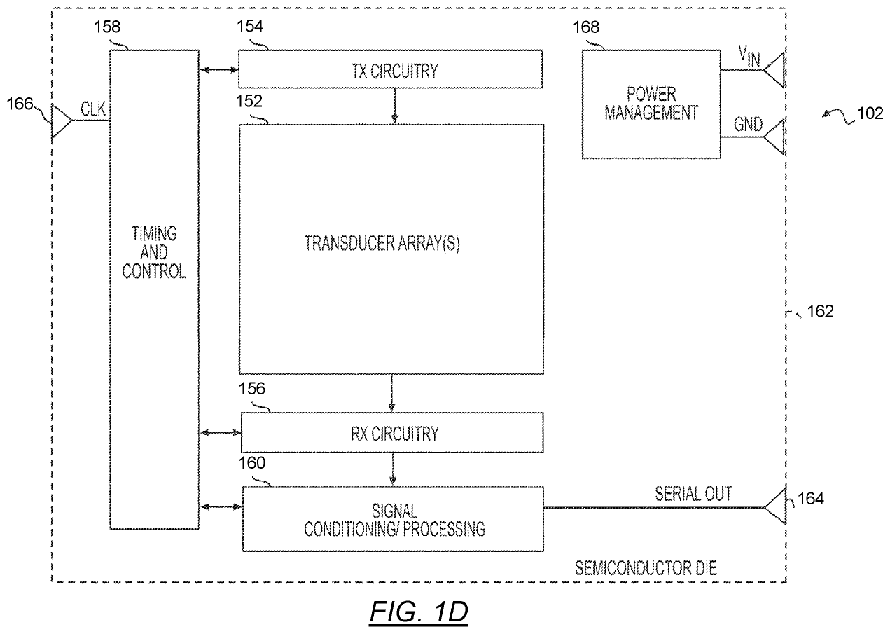

FIG. 1A shows an ultrasound system (100) in accordance with one or more embodiments. The ultrasound system (100) includes an ultrasound device (102) that is communicatively coupled to a processing device (204) by a communication link (112). Each of these components is described in further detail below.

The ultrasound device (102) is configured to obtain an ultrasound data by emitting acoustic (e.g., ultrasonic) waves into a subject (101) and detecting reflected signals from different tissue interfaces. The amplitude and phase of the reflected signal may be analyzed to identify various properties of the tissue(s) and/or interface(s) through which the acoustic wave has traveled (e.g., density of the tissue). The ultrasound device (102) may be configured to transmit raw ultrasound data, processed ultrasound images, or any combination thereof to the processing device (204). Components of the ultrasound device (102) are discussed in further detail below with respect to FIG. 1D.

The ultrasound device (102) may be implemented in any of a variety of ways. For example, the ultrasound device (102) may be implemented as a handheld device (102a) (as shown in FIG. 1B) that is controlled by a POCUS user and pressed against the subject (101). In one or more embodiments, the ultrasound device (102) may be implemented as a patch (as shown in FIG. 1C) that is attached to the subject (101) and remotely controlled by the POCUS user. Further still, in one or more embodiments, the ultrasound device (102) may include a plurality of networked devices (e.g., a plurality of patches (102b), a handheld device (102a) in conjunction with one or more patches (102b), or any combination thereof).

The ultrasound device (102) may transmit data to the processing device (204) using a communication link (112). The communication link (112) may be a wired or wireless communication link. In one or more embodiments, the communication link (112) may be implemented as a cable such as a Universal Serial Bus (USB) cable or another appropriate cable that is configured to exchange information and/or power between the processing device (204) and the ultrasound device (102). In other embodiments, the communication link (112) may be a wireless communication link such as a BLUETOOTH, WiFi, or ZIGBEE wireless communication link.

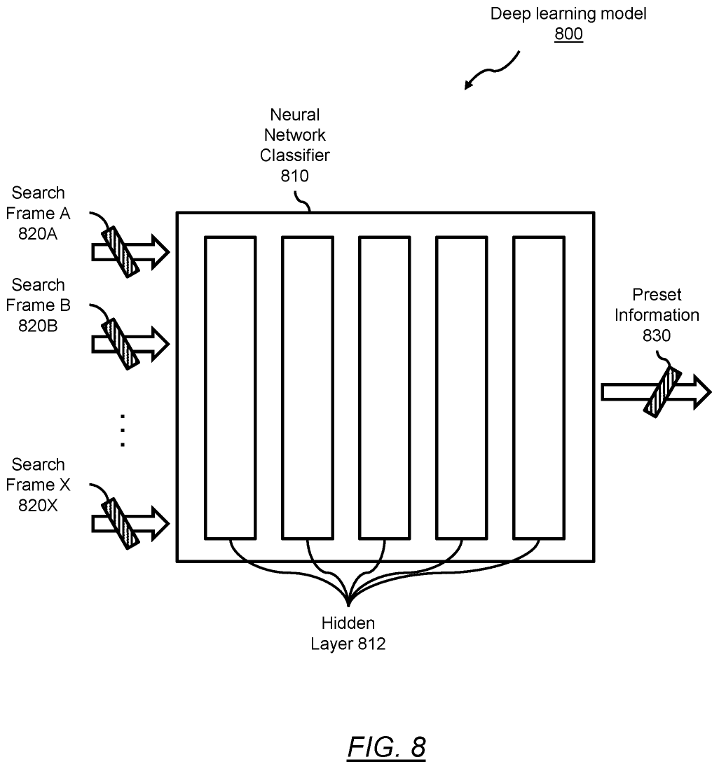

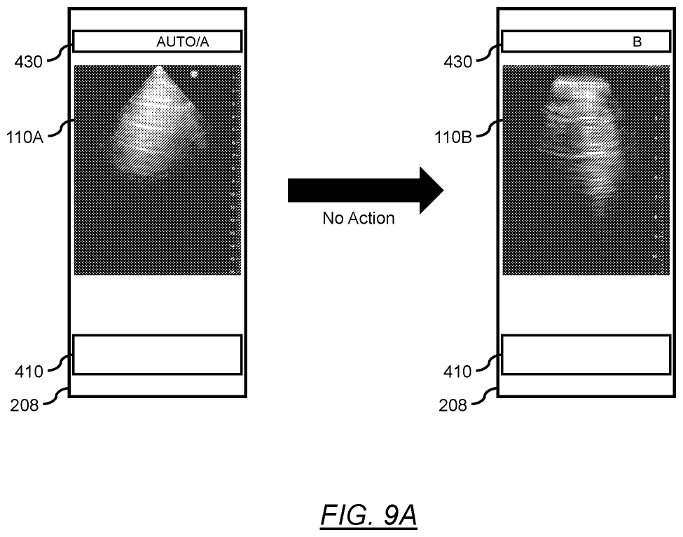

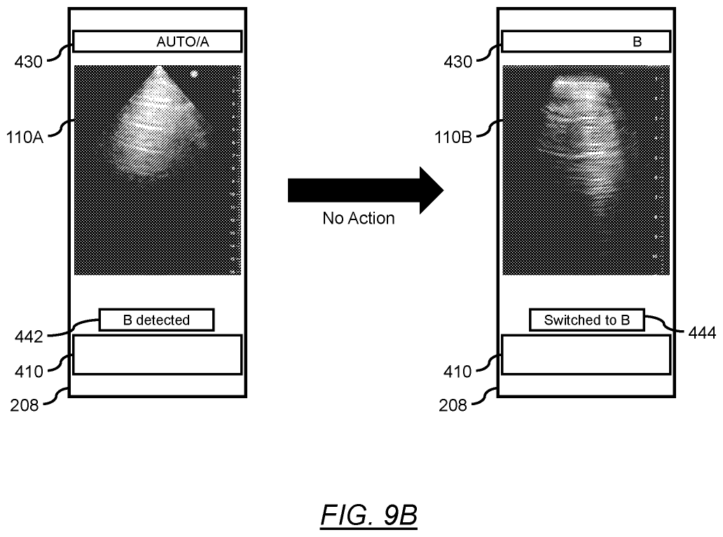

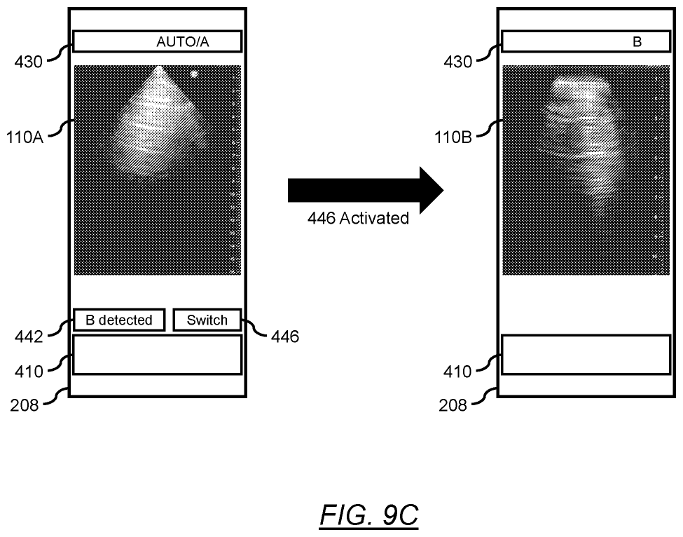

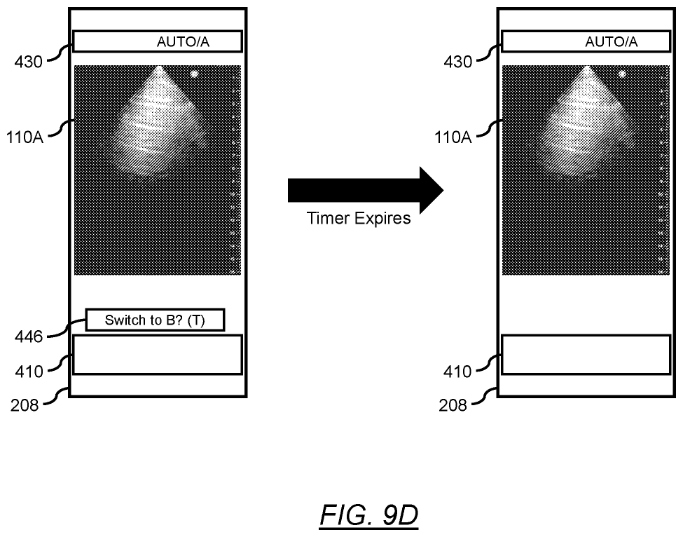

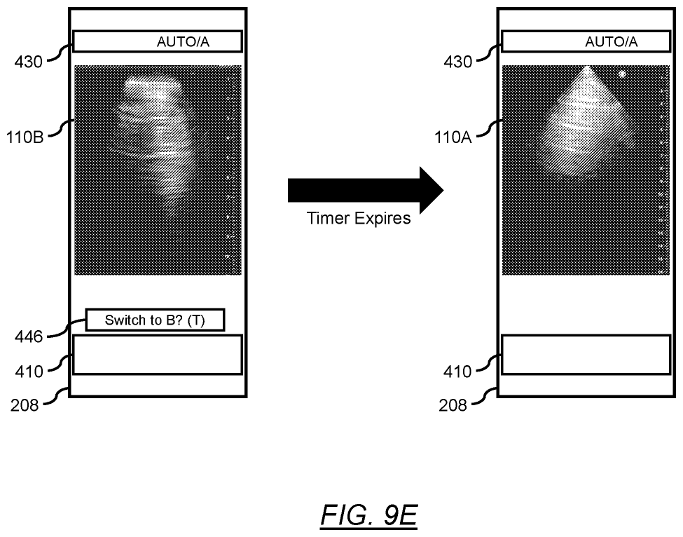

The processing device (204) controls the ultrasound device (102) and processes ultrasound data received from the ultrasound device (102). The processing device (204) may be configured to generate an ultrasound image (110) on a display screen (208) of the processing device (204). The processing device (204) further includes a user interface, described in further detail below with respect to FIGS. 9A-9E, that is displayed on the display screen (208) and provides the operator with controls and instructions (e.g., images, videos, or text) to assist a user in collecting clinically relevant ultrasound images. For example, the user interface may provide information (e.g., guidance information) prior to scanning the subject (101). In addition, the user interface may provide guidance or suggestions to the POCUS user during scanning of the subject (101). The processing device (204) may provide control options (e.g., scanning presets) and/or operating modes for the ultrasound device (102) based on anatomical features detected during scanning of the subject (101).

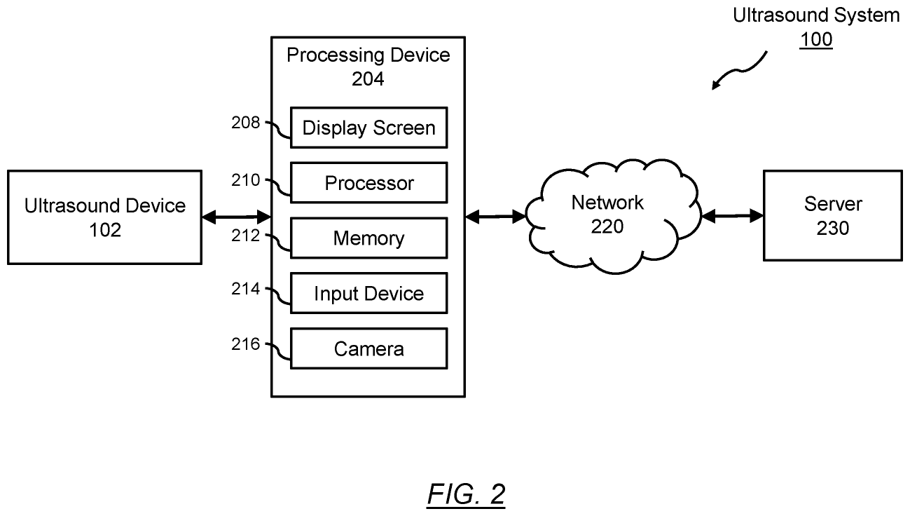

In one or more embodiments, the processing device (204) may be implemented as a mobile device (e.g., a mobile smartphone, a tablet, or a laptop) with an integrated display (208), as shown in FIG. 1A. In other examples, the processing device (204) may be implemented as a stationary device such as a desktop computer. The processing device (204) is discussed in further detail below with respect to FIG. 2.

FIG. 1B shows a handheld ultrasound probe (102a) in accordance with one or more embodiments. The handheld ultrasound probe (102a) may correspond to the ultrasound device (102) in FIG. 1A. The handheld ultrasound probe (102a) may include a wired communication link (112) that communicates with the processing device (204). For example, one or more non-limiting embodiments may have a cable for wired communication with the processing device (204), and have a length about 100 mm-300 mm (e.g., 175 mm) and a weight about 200 g-500 g (e.g., 312 g). In one or more embodiments, the handheld ultrasound probe (102a) may be wirelessly connected to the processing device (204). As such, one or more embodiments may have a length of about 140 mm and a weight of about 265 g. It will be appreciated that the handheld ultrasound probe (102a) may have any suitable dimension and weight.

FIG. 1C shows a wearable ultrasound patch (102b) in accordance with one or more embodiments. The wearable ultrasound patch (450) may be coupled to the subject (101) with an adhesive and/or coupling medium (e.g., ultrasound gel). The wearable ultrasound patch (102b) may include a wired communication link (112) that communicates with the processing device (204). In one or more embodiments, the wearable ultrasound patch (102b) may be wirelessly connected to the processing device (204).

While FIGS. 1B-1C show examples of the ultrasound device (102), it will be appreciated that other form factors are possible without departing from the scope of the present disclosure. For example, in other embodiments, the ultrasound device (102) may be in the form factor of a pill that is inserted into (e.g., swallowed by) the subject (101). The pill may be configured to wirelessly transmit ultrasound data to the processing device (204) for processing.

FIG. 1D shows a schematic of an ultrasound device (102), in accordance with one or more embodiments. The ultrasound device (102) may include one or more of each of the following: transducer arrays (152), transmit (TX) circuitry (154), receive (RX) circuitry (156), a timing and control circuit (158), a signal conditioning/processing circuit (160), a power management circuit (168). Each of these components is described in further detail below.

In FIG. 1D, all of the illustrated components are formed on a single semiconductor die (162) where the ultrasound device (102) may include one or more the semiconductor dies (162). However, in one or more embodiments, one or more of the illustrated components may be disposed on a separate semiconductor die (162) or on a separate device. Alternatively, one or more of these components may be implemented in a digital signal processing (DSP) chipset, a field programmable gate array (FPGA) in a separate chipset, or a separate application specific integrated circuitry (ASIC) chipset.

The transducer array (152) includes a plurality of ultrasonic transducer elements that transmit and receive ultrasonic signals. An ultrasonic transducer may take any forms (e.g., a capacitive micromachined ultrasonic transducer (CMUT), or a piezoelectric micromachined ultrasonic transducers (PMUT)), and embodiments of the present invention do not necessitate the use of any specific type or arrangement of ultrasonic transducer elements. A CMUT may include a cavity formed in a complementary metal-oxide semiconductor (CMOS) wafer with a membrane that overlays and/or seals the cavity (i.e., the cavity structure may be provided with electrodes to create an ultrasonic transducer cell). In one or more embodiments, one or more components of the ultrasound device (102) may be included in integrated circuitry of the CMOS wafer (i.e., the ultrasonic transducer cell and CMOS wafer may be monolithically integrated).

In one or more embodiments, the transducer array (152) may include ultrasonic transducer elements arranged in a one-dimensional or a two-dimensional distribution. The distribution may be an array (e.g., linear array, rectilinear array, non-rectilinear array, sparse array), a non-array layout (e.g., sparse distribution), and any combination thereof.

In a non-limiting example, the ultrasonic transducer array (152) may include between approximately 6,000-10,000 (e.g., 8,960) active CMUTs on the chip, forming an array of hundreds of CMUTs by tens of CMUTs (e.g., 140×64). The CMUT element pitch may be between 150-250 um, such as 208 μm, and thus, result in the total dimension of between 10-50 mm by 10-50 mm (e.g., 29.12 mm×13.312 mm).

In some embodiments, the frequency range of a transducer unit (152) may be greater than or equal to 1 MHz and less than or equal to 12 MHz to allow for a broad range of ultrasound applications without changing equipment (e.g., changing of the ultrasound units for different operating ranges). For example, the broad frequency range allows a single unit to perform medical imaging tasks including, but not limited to, imaging a liver, kidney, heart, bladder, thyroid, carotid artery, lower venous extremity, and performing central line placement, as shown in the examples of Table 1.

| Organ | Frequencies | Depth (up to) | |
|---|---|---|---|
| Liver/Right Kidney | 2-5 MHz | 15-20 | cm |
| Cardiac (adult) | 1-5 MHz | 20 | cm |
| Bladder | 2-5 MHz; | 10-15 | cm; |
| | 3-6 MHz | 5-10 | cm |
| Lower extremity venous | 4-7 MHz | 4-6 | cm |
| Thyroid | 7-12 MHz | 4 | cm |
| Carotid | 5-10 MHz | 4 | cm |
| Central Line Placement | 5-10 MHz | 4 | cm |

TABLE 1: Illustrative depths and frequencies at which an ultrasound device (102) in accordance with one or more embodiments may image a subject (101).

The TX circuitry (154) generates signal pulses that drive the ultrasonic transducer elements of the ultrasonic transducer array (152). In one or more embodiments, the TX circuitry (154) includes one or more pulsers that each provide a signal pulse to individual ultrasonic transducer elements or one or more groups of ultrasonic transducer elements of the ultrasonic transducer array (152).

The RX circuitry (156) receives and processes the electronic signals generated by the individual ultrasonic transducer elements of the ultrasonic transducer array (152).

In one or more embodiments, the individual ultrasonic transducer elements of the ultrasonic transducer array (152) may be connected to one or both of the TX circuitry (154) and RX circuitry (156). For example, the ultrasonic transducer elements may be: limited to transmitting acoustic signals; limited to receiving acoustic signals; or perform both transmission and receiving of acoustic signals. In one or more embodiments, the ultrasonic transducer elements of the ultrasonic transducer array (152) may be formed on the same chip as the electronics of the TX circuitry (154) and/or RX circuitry (156). The ultrasonic transducer arrays (152), TX circuitry (154), and RX circuitry (156) may be integrated in a single probe device.

The timing and control circuit (158) generates timing and control signals that synchronize and coordinate the operation of the other elements in the ultrasound device (102). In one or more embodiments, the timing and control circuit (158) is driven by a clock signal CLK supplied to an input port (166). In one or more embodiments, two or more clocks of different frequencies may be separately supplied to the timing and control circuit (158). Furthermore, the timing and control circuit (158) may divide or multiply a clock signal to drive other components on the die (162).

In a non-limiting example, a 1.5625 GHz or 2.5 GHz clock may be used to drive a high-speed serial output device (164) connected to the signal conditioning/processing circuit (160) and a 20 Mhz or 40 MHz clock used to drive digital components on the die (162).

The power management circuit (168) manages power consumption within the ultrasound device (102) and converts one or more input voltages VIN from an off-chip source into the appropriate voltages required to operate the components of the ultrasound device (102). In one or more embodiments, a single voltage (e.g., 12 V, 80 V, 100 V, 120 V) may be supplied to the ultrasound device (102) (e.g., via a cable of a wired communication link (112) in a wired device, via a battery in a wireless device) and the power management circuit (168) may include a DC/DC converter to step the voltage up or down, as necessary. Alternatively, multiple voltages may be supplied to the power management circuit (168) for regulation and distribution to the components of the ultrasound device (102).

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

In the example shown, one or more output ports (164) may output a high-speed serial data stream generated by one or more components of the signal conditioning/processing circuit (160). Such data streams may be, for example, generated by one or more USB 3.0 modules, and/or one or more 10 GB, 40 GB, or 100 GB Ethernet modules, integrated on the die (162). It is appreciated that other communication protocols may be used for the output ports (164).

In some embodiments, the signal stream produced on output port (164) can be provided to a processing device (204) (e.g., computer, tablet, smartphone, etc.) for the generation and/or display of two-dimensional, three-dimensional, and/or tomographic images. In some embodiments, the signal provided at the output port (164) may be ultrasound data provided by the one or more beamformer components or auto-correlation approximation circuitry, where the ultrasound data may be used by the processing device (204) for displaying the ultrasound images (110). In embodiments in which image formation capabilities are incorporated in the signal conditioning/processing circuit (160), even relatively low-power devices, such as smartphones or tablets which have only a limited amount of processing power and memory available for application execution, can display images using only a serial data stream from the output port (164). As noted above, the use of on-chip analog-to-digital conversion and a high-speed serial data link to offload a digital data stream is one of the features that helps facilitate an “ultrasound on a chip” solution according to some embodiments of the technology described herein.

In general, ultrasound devices (102) in accordance with one or more embodiments may be used in various imaging and/or treatment (e.g., High-Intensity Focused Ultrasound (HIFU)) applications. The examples described herein should not be viewed as limiting.

In one illustrative implementation, for example, an ultrasound device (102) including an N×M planar or substantially planar array of CMUT elements may acquire an ultrasound image of the subject (101) by energizing some or all of the ultrasonic transducer elements in the ultrasonic transducer array (152) during one or more transmit phases, and receiving and processing signals generated by some or all of the ultrasonic transducer elements in the ultrasonic transducer array (152) during one or more receive phases. In other implementations, some of the elements in the ultrasonic transducer array (152) may be used only to transmit acoustic signals and other elements in the same ultrasonic transducer array (152) may be used only to receive acoustic signals. Moreover, in some implementations, a single imaging device may include a P×Q array of individual devices (e.g., semiconductor dies (162)), or a P×Q array of individual N×M planar arrays of CMUT elements, that are operated in parallel, sequentially, or according to any appropriate timing scheme that allows data to be accumulated.

While FIG. 1D shows an example configuration of components in the ultrasound device (102), other configurations may be used without departing from the scope of the disclosure. For example, various components in FIG. 1D may be combined to in a single component (e.g., a DSCP chipset, an FPGA chipset, an ASIC chipset, or any programmable processing device). In addition, the functionality of each component described above may be shared among multiple components or performed by a different component than described above. In addition, each component may be utilized multiple times (e.g., in serial, in parallel, in different locations) to perform the functionality of the claimed invention.

FIG. 2 shows a schematic of an ultrasound system (100) in accordance with one or more embodiments. In one or more embodiments, the ultrasound system (100) includes a processing device (204) that communicates with one or more servers (230) via a network (220). The processing device (204) is further communicatively coupled to the ultrasound device (102) (e.g., via a wireless or wired communication link (112)) to process the ultrasound data from the ultrasound device (102). For example, the processing device (204) may perform some or all of the correlation of ultrasound signals transmitted or received by the ultrasound transducer (152).

The processing device (204) includes a display screen (208), a processor (210), and a memory (212). In one or more embodiments, the processing device (204) may further include an input device (214) and/or a camera (216). The display screen (208), the input device (214), the camera (216), and/or any other input/output interfaces (e.g., speaker, connect device or peripheral apparatus) may be communicatively coupled to and controlled by the processor (210). Each of these components is described in further detail below.

The display screen (208) displays images and/or videos (e.g., ultrasound imagery). The display screen (208) may include a liquid crystal display (LCD), a plasma display, an organic light emitting diode (OLED) display, or any combination of appropriate display devices.

The processor (210) includes one or more processing units (e.g., central processing unit (CPU), graphics processing unit (GPU), tensor processing unit (TPU)) that controls the ultrasound device (102) (e.g., sets operating parameters, controls components in FIG. 1D) and processes ultrasound data from the ultrasound device (102). In one or more embodiments, the processor (210) may include specially-programmed and/or special-purpose hardware such as an ASIC chip. For example, an ASIC included in the processor (210) may be specifically designed for machine learning (e.g., a deep learning model) and may be employed to accelerate the inference phase of a neural network (described in further detail below with respect to FIG. 8).

The processor (210) is configured to control the acquisition and processing of ultrasound data with the ultrasound device (102). The ultrasound data may be processed in real-time during a scanning session and displayed to the POCUS user via the display screen (208). In one or more embodiments, the ultrasound image may be updated at a rate of at least 5 Hz, at least 10 Hz, at least 20 Hz, at a rate between 5 and 60 Hz, or at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

In some embodiments, the processing device (204) may be configured to perform various ultrasound operations using the processor (210) (e.g., one or more computer hardware processors) and one or more articles of manufacture that include non-transitory computer readable media (CRM). To perform certain of the processes described herein, the processor (210) may execute one or more instructions stored in one or more non-transitory CRM (e.g., the memory (212)).

The memory (212) includes one or more storage elements (e.g., a non-transitory CRM) to, for example, store instructions that may be executed by the processing (210) and/or store all or any portion of the ultrasound data received from the ultrasound device (102). The processor (210) may control writing data to and reading data from the memory (212) in any suitable manner. In one or more embodiments, the memory (212) stores presets for the ultrasound system (100), where each preset includes one or more modes used to control the ultrasound device (102) and one or more tools to analyze ultrasound data from the ultrasound device (102).

The input device (214) includes one or more devices capable of receiving input from a POCUS user and transmitting the input to the processor (210). For example, the input device (214) may include a keyboard, a mouse, a microphone, touch-enabled sensors on the display screen (208), a camera (216), and/or a microphone.

The camera (216) detects light to form an image. The camera (216) may be on any side of the processing device (204) (e.g., on the same side as the display screen (208)). In one or more embodiments, the camera (216) acquires images as medical information of the subject (101) and to aid in navigation of the ultrasound device (102). For example, an image of the subject (101) and the ultrasound device (102) may be used to generate location information or to determine what anatomical region of the subject is being scanned.

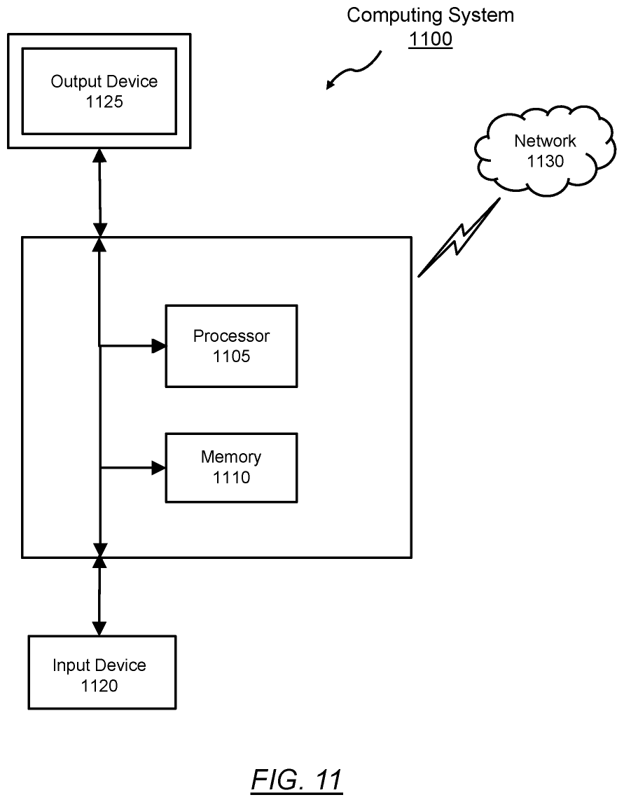

It should be appreciated that the processing device (204) may be implemented in any of a variety of ways. For example, the processing device (204) may be implemented as a handheld device such as a mobile smartphone or a tablet. Thereby, a POCUS user may be able to operate an ultrasound device (102) with one hand and hold the processing device (204) with another hand. In other examples, the processing device (204) may be implemented as a portable device that is not a handheld device, such as a laptop. In yet other examples, the processing device (204) may be implemented as a stationary device such as a desktop computer. In general, the processing device (204) may be implemented on virtually any type of computing system (1100), regardless of the platform being used, as described in further detail below with respect to FIG. 11.

The network (220) may be a wired connection (e.g., via an Ethernet cable) and/or a wireless connection (e.g., over a WiFi network) that connects the processing device (204) to another computing device. For example, the processing device (204) may thereby communicate with (e.g., transmit data to or receive data from) the one or more servers (230) over the network (220). For example, a party may provide from the server (234) to the processing device (204) processor-executable instructions for storing in one or more non-transitory computer readable storage media (e.g., the memory (212)) which, when executed, may cause the processing device (204) to perform ultrasound processes.

While FIG. 2 shows an example configuration of components, other configurations may be used without departing from the scope of the disclosure. For example, the ultrasound system (100) may include fewer or more components than shown. In one or more embodiments, the processing device (204) and ultrasound device (102) may include fewer or more components than shown. Further, various components in FIG. 2 may be combined to create a single component (e.g., display screen (208) and input device (214)). In addition, the functionality of each component described above may be shared among multiple components or performed by a different component than that described above (e.g., in addition to the core functions, additionally perform each other's functions). In one or more embodiments, the processing device (204) may be part of the ultrasound device (102). In addition, each component may be utilized multiple times (e.g., in serial, in parallel, distributed over a network (220)) to perform the functionality of the claimed invention.

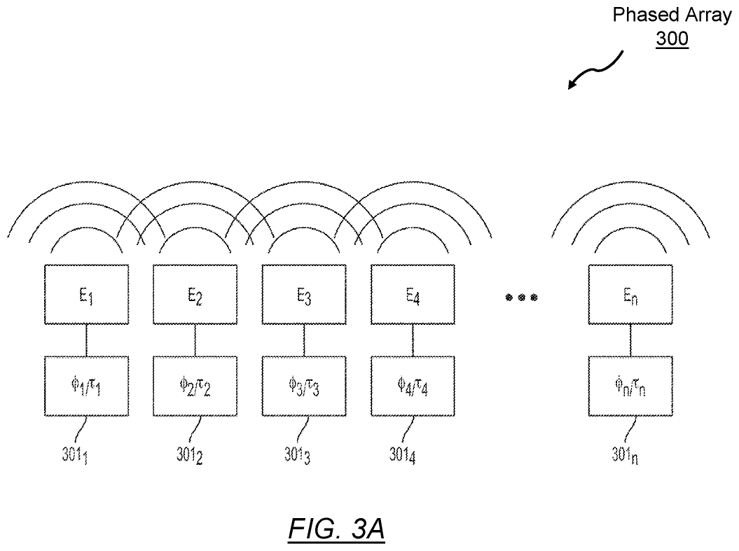

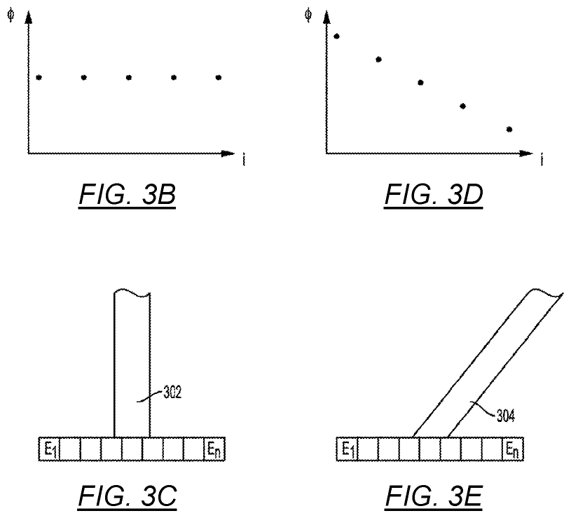

FIG. 3A shows a phased array (300) of the ultrasound device (102), in accordance with one or more embodiments. An ultrasound device (102) comprising a plurality of ultrasound transducers Eᵢ (i=1, 2 . . . N), where N may be any appropriate number of elements, may be operated as a phased array (300). An ultrasound beam emitted from the ultrasound device (102) may be oriented by adjusting the phases of a plurality of transducers. The ultrasound transducers may be arranged as a two-dimensional array, as a one-dimensional array, sparsely arranged, or otherwise arranged in any predetermined spatial distribution.

Each ultrasound transducer Eᵢ is configured to receive a drive signal having a certain phase and a certain time delay based upon the predetermined spatial distribution. For example, ultrasound transducer E₁ is driven by a signal having a phase Φ₁ and a delay τ₁, ultrasound transducer E₂ is driven by a signal having a phase Φ₂ and a delay τ₂, ultrasound transducer E₃ is driven by a signal having a phase Φ₃ and a delay τ₃, ultrasound element E₄ is driven by a signal having a phase Φ₄ and a delay τ₄, and ultrasound transducer E_{N} is driven by a signal having a phase Φ_{N} and a delay τ_{N}. The phase and the delay of the drive signals may be controlled using signal drivers 301₁, 301₂, 301₃, 301₄, and 301_{N} (e.g., the TX circuitry (154) in FIG. 1D). The signal drivers may comprise phase shifters and/or adjustable time delay units. According to the manner in which the various phases are controlled relative to one another, the individual ultrasound waves emitted by the ultrasound elements may experience different degrees of interference (e.g., constructive interference, destructive interference, or any suitable value in between).

In some embodiments, the phases (i and/or time delays Φᵢ may be controlled to cause the ultrasound waves to interfere with one another so that the resulting waves add together to increase the acoustic beam in a desired direction. The phases Φᵢ and/or time delays τᵢ may be controlled with respective signal drivers, which may be implemented for example using transistors and/or diodes arranged in a suitable configuration. In at least some of the embodiments in which the ultrasound elements are disposed on a semiconductor substrate, the signal drivers may be disposed on the same semiconductor substrate.

FIGS. 3B-3E show an example of an operation of a phased array in accordance with one or more embodiments.

FIG. 3B is a plot illustrating the phase of the signal with which each transducer Ei is driven. In the example, the ultrasound transducers are driven with uniform phases. As a result, the acoustic beam (302) is mainly directed along the perpendicular to the plane of the ultrasound device, as illustrated in FIG. 3C.

In the example of FIG. 3D, the ultrasound transducers are driven with phases arranged according to a linear relationship. As a result, the angled acoustic beam (304) is offset relative to the perpendicular to the plane of the ultrasound device, as illustrated in FIG. 3E.

While not shown, the phases may be arranged in other manners. For example, the ultrasound transducers may be driven with phases arranged according to a quadratic relationship, which may result in an acoustic beam that converges. Any other suitable phase relationship may be used.

In some embodiments, the phases may be adjusted to produce steering within a 3D field-of-view. This may be accomplished for example by adjusting azimuth and elevation of the emissions. Accordingly, the acoustic beam may be steered through an entire volume at a desired angle, with respect to the direction that is normal to the aperture of the transducer array.

While the above discussion is for the transmit phase, similar methods may be applied during the receive phase. Accordingly, beamforming may involve beamforming when transmitting, when receiving, or both.

In one or more embodiments, the memory (212) stores a plurality of presets (i.e., operational modes) that each include one or more modes used to control the ultrasound device (102) (e.g., beamforming parameters, scan pattern, ultrasonic frequency, imaging depth, virtual aperture size, virtual aperture offset, imaging rate, imaging type, timing information, synchronization information). In addition, each preset includes one or more tools to analyze ultrasound data (e.g., imaging tools, analysis tools, measurement tools, deep learning algorithms).

A non-limiting list of example presets include Cardiac Standard, Cardiac Deep, Coherence, Pediatric Cardiac, Abdomen, Bladder, Fast, Aorta/Gallbladder, Pediatric Abdomen, Musculoskeletal (MSK), Vascular access, Carotid, Small Parts, Thyroid, Lung, Pediatric Lung. Each preset may be optimized for scanning the indicated anatomical feature by tuning the one or more parameters (e.g., frequencies and depth in Table 1), and enabling/disabling one or more tools.

In one or more embodiments, the processing device (204) may be configured to utilize any or all of the modes and tools specified by all of the presets, but only the modes and tools that are specified by the actively selected preset are enabled for use. In other words, each preset may guide a POCUS user by limiting the capabilities of the processing device (204) to the appropriate settings for a given ultrasound scan.

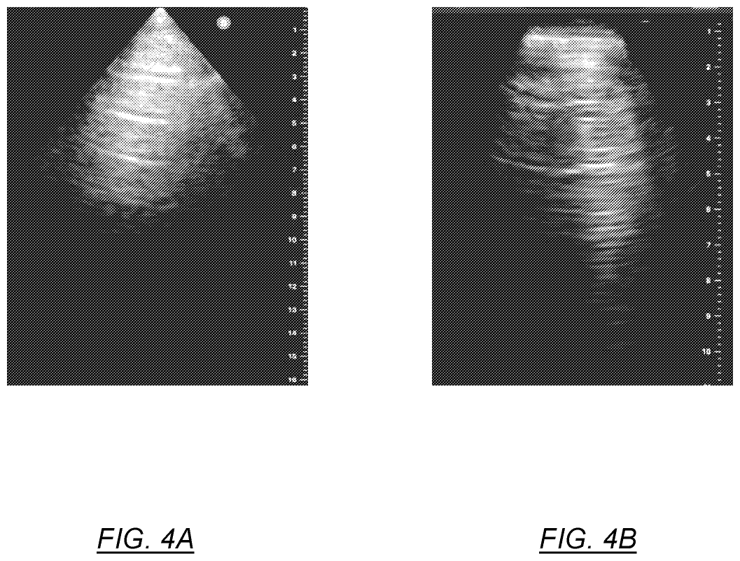

FIGS. 4A-4B show examples of ultrasound images (110) acquired with different presets in accordance with one or more embodiments. Specifically, FIG. 4A shows an ultrasound image (110A) of a lung in the subject (101) while using a “Cardiac Preset” and FIG. 4B shows an ultrasound image (110B) of the same region in the subject (101) while using a “Lung Preset.” Because the two different presets use different parameters (e.g., frequency and scan depth), different ultrasound images (110A, 110B) are presented to the POCUS user. Furthermore, the different presets may provide the POCUS user with different tools (e.g., pulse rate detection for “Cardiac Preset” versus breathing rate detection for “Lung Preset”) to provide or collect more relevant diagnostic information.

Embodiments of the present invention are directed to an “Auto Mode” that automatically determines a preset that is appropriate for an ultrasound scan being performed. The “Auto Mode” may automatically switch to the determined preset or may suggest that the POCUS user switches to the determined preset.

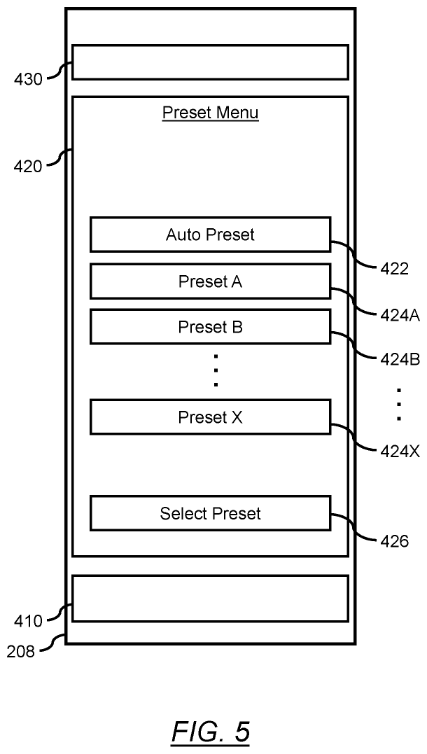

FIG. 5 shows an example of a user interface of the processing device (204) in accordance with one or more embodiments. During an ultrasound scan, the processing device (204) may present the POCUS user with a user interface (UI) that includes one or more of the following elements: a control interface (410), which can open a preset menu (420); and a status interface (430). Each of these UI elements is described in further detail below.

The control interface (410) presents the POCUS user with one or more control elements (e.g., buttons, icons, menus, sliders) for interacting with the processing device (204). For example, the processing device (204) may run an application (e.g., an app) with a UI including one or more of the following buttons: a preset button (e.g., controls the preset menu (420)); a multi-use button (e.g., controls a tool or function in the current preset mode); a capture button (e.g., controls acquisition/recording of an image or a video of the ultrasound image (110)); an action button (e.g., controls a menu for any other actions available to the POCUS user). In one or more embodiments, the control interface (410) may be split into multiple sections (e.g., on the bottom of the display screen (208) and on the top of the display screen (208)). In one or more embodiments, the control elements of the control interface (410) may not be visibly displayed on the display screen (208) (e.g., controls triggered by voice recognition from a microphone, controls triggered by image analysis from a camera (216)). Interacting with the control elements of the control interface (410) may be performed by touching a touchscreen interface of the display screen (208) or any method that the processing device (204) is configured to accept user input. In other words, the control interface (410) may include any number and arrangement of control elements for the POCUS user to control the processing device (204).

The preset menu (420) presents the POCUS user with one or more control elements to select a preset for the processing device (204). In one or more embodiments, the preset menu (420) includes a series of buttons corresponding to each of the presets that the processing device (204) is capable of operating. For example, the preset menu (420) may include an “Auto Mode” button 422 and a series of buttons 424 for each of the available presets (e.g., Preset A, Preset B, . . . , Preset X). In one or more embodiments, the preset menu (420) may further include a “Select” button 226 to confirm the selection before the processing device (204) switches the current preset.

Status interface (430) presents the POCUS user with one or more status elements (e.g., visual indicator, label, icon, amination, light, audio, etc.) to convey information about the status of the processing device (204). In one or more embodiments, the status interface (430) includes a visual indicator (e.g., text label) of the current preset that is in use. Additional status elements may present relevant information for the current scan (e.g., frequency, imaging rate, battery status). In one or more embodiments, the status elements of the status interface (430) may not be visibly displayed on the display screen (208) (e.g., audio played from a speaker). In other words, the status interface (430) may include any number and arrangement of status elements to inform the POCUS user.

While UI is described with respect to a limited number of examples, other configurations of the UI may be used without departing from the scope of the disclosure. For example, the UI may include fewer or more elements than shown. Further, various elements of the UI may be combined to create a single UI element (e.g., part or all of the preset menu (420) integrated into the control interface (410)) or separated into multiple elements (e.g., status interface (430) divided into a plurality of separate elements to improve visual impact). In addition, the functionality of each UI element described above may be shared among multiple UI elements or performed by a different UI element than described above (e.g., in addition to the core functions, additionally perform each other's functions). In addition, each UI element may be utilized multiple times to perform the functionality of the claimed invention.

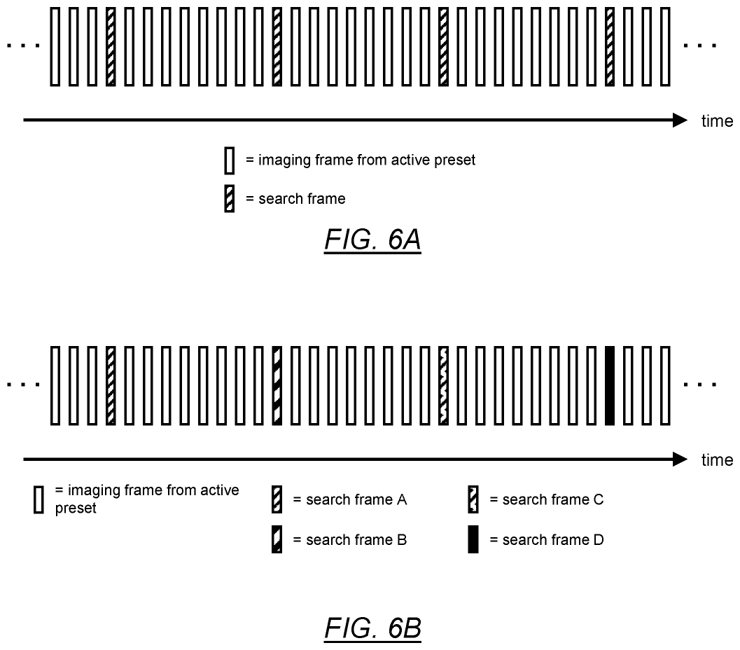

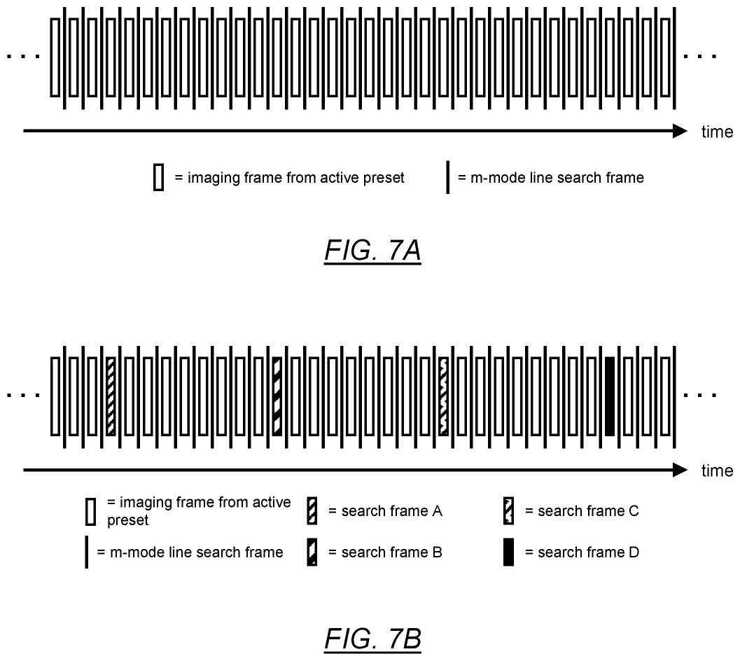

As discussed above, embodiments of the present invention are directed to an “Auto Mode” that automatically determines a target preset for an ultrasound scan. To perform the determination, the processing device (204) generates ultrasound images using ultrasound data from the ultrasound probe from an ultrasound device (102) and identifies an anatomical feature in the ultrasound images using a deep learning model (discussed in further detail below with respect to FIG. 8). To identify the anatomical feature, a processing device (204) utilizes a frame acquisition technique in accordance with one or more embodiments shown in FIGS. 6A-7B below, to obtain search frames for analysis while minimally impacting the POCUS user's ability to use the processing device (204) to view real-time images from the ultrasound device (102).

In one or more embodiments, the “Auto Mode” is the default setting for the processing device (204). In one or more embodiments, the “Auto Mode” is the default setting for a category of users of the processing device (204) (e.g., users with less experience).

FIGS. 6A-6B show schematics of frame acquisition techniques with a search frame in accordance with one or more embodiments.

Once the “Auto Mode” is initialized (e.g., opened by default, selected by the POCUS user), the processing device (204) operates the ultrasound device (102) using a first preset to acquire ultrasound data. In one or more embodiments, the first preset may include a mode and/or a tool specified by the “Auto Mode.” Alternatively, the first preset may be selected by the POCUS user (e.g., selected from a predetermined preset button (424) in addition to the “Auto Mode” button (422), a preset in use by the POCUS user prior to the “Auto Mode” being initialized).

Using the first preset, the processing device (204) generates a first portion of ultrasound images using ultrasound data from the ultrasound device (102). The first portion of the ultrasound images are imaging frames that are shown to the POCUS user on the display screen (208) of the processing device (204). For example, the imaging frames are shown in real-time to the POCUS user (e.g., as in a typical scan).

As shown in FIG. 6A, when the “Auto Mode” is active, the processing device (204) generates a second portion of ultrasound images using ultrasound data from the ultrasound device (102). The second portion of the ultrasound images are search frames that are used as inputs to a deep learning model for identification of any anatomical feature during the scan. In one or more embodiments, the search frames are not shown to the POCUS user on the display screen (208) of the processing device (204).

In one or more embodiments, the search frames and the imaging frames are two-dimensional ultrasound images. However, in one or more embodiments, the search frames may be one-dimensional line scan images or a combination of two-dimensional ultrasound images and one-dimensional line scan images. Therefore, the deep learning model that identifies anatomical features in the search frames may be a neural network classifier trained with two-dimensional ultrasound images and/or one-dimensional line scan images based on type of images in the search frames.

For example, for two-dimensional ultrasound images, the neural network classifier may be trained to identify anatomical features based on one or more image recognition algorithms (i.e., the neural network classifier is trained with ultrasound images generated using parameters of a two-dimensional scan mode). Alternatively, for one-dimensional line scan images (which can be acquired at much higher rates due to smaller scan size), the deep learning model may be a neural network classifier trained to identify anatomical features based on temporal dynamics in the line scan images (i.e., the neural network classifier is trained with time-varying line scan images generated using parameters of a line scan mode).

In one or more embodiments, the search frames are time-interleaved with the imaging frames. For example, the search frames may be evenly distributed between imaging frames such that the frame rate of imaging frames shown on the display screen is not significantly degraded. The search frames are time-interleaved within the imaging frames with a fixed periodicity, an aperiodic distribution, a random distribution, or any combination thereof.

In one or more embodiments, the search frames may be acquired with the first preset. In other words, a preset for the search frames (i.e., a search preset) is the same as the first preset (e.g., the search frames are simply selected from imaging frames). Because the first preset may be any one of the presets available on the processing device, the neural network classifier according to one or more embodiments is trained with ultrasound images generated using parameters of each of the presets stored in the memory. For example, in a processing device with four available presets, the neural network classifier would require training images of different anatomical features in each of the four available presets to identify an observed anatomical when the user selects any one of the four available presets as the first preset used in “Auto Mode.”

In one or more embodiments, the search frames may be acquired with a search preset that is different from the first preset. In other words, while a POCUS user may select any available preset as the first preset to image the subject (101) (i.e., viewing imaging frames acquired with the first preset on the display screen (208)), the processing device (204) acquires the search frames using a predetermined search preset. In this non-limiting example, the search frames are generated only using the predetermined search preset. Therefore, the neural network classifier can be trained with a relatively smaller set of ultrasound images that are generated using the specified parameters of the predetermined search preset.

As shown in FIG. 6B, in one or more embodiments, the search preset includes a plurality of pillar presets that are different from the first preset. In other words, the search frames include ultrasound images acquired using each of the different pillar presets and the neural network classifier is trained with ultrasound images generated using parameters of each of the different pillar presets. Using a wider range of acquisition parameters improves the chance of recognizing anatomical features in the subject (101). Furthermore, in one or more embodiments, the pillar presets include predetermined parameters that do not change over time while the default presets available to the POCUS user may be updated over time (e.g., updated software releases for the processing device (204)). By using fixed and predetermined pillar presets, retraining the neural network classifier is unnecessary, even when the default presets available to the POCUS user are updated.

In one or more embodiments, each of the plurality of pillar presets utilizes a different combination of frequency and imaging depth parameters. The pillar presets may be designed to approximate different groups of presets programmed into the processing device (204) to simplify the space of parameters being used for the search frames.

For example, the pillar preset may be based on depth (e.g., 3 cm, 6 cm, 12 cm, 18 cm, etc.), transmit frequency, beam spread scaling, or any combination thereof. In one or more non-limiting examples, the first pillar preset may have maximum frequency and minimum depth parameters while the last pillar preset may have minimum frequency and maximum depth parameters (the remaining pillar presets distributed between the minimum/maximum settings).

In one or more non-limiting embodiments, the plurality of different pillar presets correspond to different anatomical regions. For example, the plurality of different pillar presets include four pillar presets corresponding to a cardiac anatomical region, an abdominal anatomical region, a musculoskeletal anatomical region, and a lung region.

FIGS. 7A-7B show schematics of frame acquisition techniques with a line search frame in accordance with one or more embodiments. As discussed above, identifying anatomical features may also be achieved based on temporal dynamics.

As shown in FIG. 7A, in one or more embodiments, to achieve sufficient time resolution for tracking temporal dynamics, the search frames may include line scan images which can be acquired at much higher rates due to the smaller scan size relative to a two-dimensional image. In other words, the search preset may include a one-dimensional line scan mode (e.g., an M-mode scan) that is different from the first preset that acquires two-dimensional images to present to the POCUS user. The resulting one-dimensional line scan search frames may be time-interleaved between every imaging frame (e.g., one or more line scan search frames acquired between every imaging frame acquired).

The deep learning model used to identify anatomical features may be a neural network classifier trained to classify temporal dynamics (e.g., heart rate determined based on periodic fluctuations in cardiac anatomical feature, breathing rate determined based on periodic fluctuations in lung anatomical feature). Accordingly, the neural network classifier may be trained with time-varying line scan images generated using parameters of the line scan mode.

As shown in FIG. 7B, in one or more embodiments, the search preset may include a combination of all of the above features to provide the widest possible search space for identifying anatomical features. In other words, in addition to the line scan images of FIG. 7A, the search preset may further include a plurality of pillar preset. In one or more embodiments, the search frames can include any combination of presets, not just the pillar presets. Accordingly, the neural network classifier described with reference to FIG. 7A may further be trained to identify anatomical features based on image recognition of the two dimensional ultrasound images in addition to temporal dynamics and may be further trained with ultrasound images generated using parameters of each of the different pillar presets.

In one or more embodiments, the processing device (204) is configured to determine an image quality of the search frames during analysis for the anatomical feature. For example, to minimize disruption in the acquisition and processing of the ultrasound data, the search frames may not be analyzed in the background when the image quality is less than a predetermined threshold. The predetermined threshold for quality may be based on any appropriate metric (e.g., resolution, stability, successful segmentation, clinical usability estimate, clipping threshold, size threshold, equipment health, etc.). In one or more embodiments, the search frames are deleted from the processing device (204) when the image quality is less than the predetermined threshold.

In one or more embodiments, the search preset (e.g., including a two-dimensional image scan mode, a one-dimensional scan mode, or multiple scan modes) utilizes a Nyquist sampling rate and no image processing such that the second portion of ultrasound images are generated faster than the first portion of images.

FIG. 8 shows a schematic of a deep learning model (800) in accordance with one or more embodiments.

Generally, the deep learning model (800) is trained to identify anatomical features in ultrasound data. In one or more embodiments, one or more machine learning algorithms are used to train a deep learning model 800 to accept search frames (820) (e.g., one or more two-dimensional ultrasound images and/or one or more one-dimensional line scan images) and to output preset information (830) (e.g., one or more target presets). In some embodiments, real, synthetic, and/or augmented (e.g., curated, or supplemented data) ultrasound images may be combined to produce a large amount of interpreted data for training the deep learning model (800).

In one or more embodiments, the deep learning model (800) includes a neural network classifier (810). The neural network classifier (810) may include one or more hidden layers 812 (e.g., convolutional, pooling, filtering, down-sampling, up-sampling, layering, regression, dropout, etc.). In some embodiments, the number of hidden layers may be greater than or less than the five layers shown in FIG. 8. The hidden layers (812) can be arranged in any order.

Each hidden layer (812) includes one or more modelling neurons. The neurons are modelling nodes or objects that are interconnected to emulate the connection patterns of the human brain. Each neuron may combine data inputs with a set of network weights and biases for adjusting the data inputs. The network weights may amplify or reduce the value of a particular data input to alter the significance of each of the various data inputs for a task that is being modeled. For example, shifting an activation function of a neuron may determine whether or not, and to what extent, an output of one neuron affects other neurons (e.g., one neuron output may be a weight value for use as an input to another neuron or hidden layer (812)). Through machine learning, the neural network classifier (810) may determine which data inputs should receive greater priority in determining one or more specified outputs of the neural network classifier (810).

While FIG. 8 shows an example configuration, other model configurations may be used without departing from the scope of the disclosure. For example, a different type of deep learning model (e.g., categorization algorithm) may be used in addition to or instead of a neural network classifier (810). Accordingly, the scope of the invention should not be limited by the model depicted in FIG. 8.

FIGS. 9A-9E show examples of a processing device (208) switching between presets in accordance with one or more embodiments.

In FIG. 9A, after detecting an anatomical marker that indicates “Preset B” (i.e., the target preset based on the identified anatomical feature) with a confidence level above a predetermined threshold, the processing device (204) automatically switches from the “Preset A” in “Auto Mode” to “Preset B.” On the display screen (208), the user interface is modified by updating the ultrasound image (110A) displayed to the POCUS user to the ultrasound image (110B) based on the parameters of “Preset B.” Furthermore, the status interface (430) is updated to reflect the new preset in use.

In FIG. 9B, after detecting an anatomical marker that indicates “Preset B” with a confidence level above a predetermined threshold, the user interface of the processing device (204) is modified by displaying a suggestion message (442) on the display screen (208) before switching from the “Preset A” in “Auto Mode” to “Preset B.” The suggestion message (442) may be any indicator (e.g., a text object, a button) that conveys the target preset for the ultrasound scan. The suggestion message (442) may be displayed on the display screen (208) for a predetermined amount of time before the ultrasound image (110A) is updated to the ultrasound image (110B). When the ultrasound image (110) is switched, in addition to the status interface (430) being updated to reflect the new preset, the processing device (204) may display a suggestion confirmation (444) on the display screen (208). The confirmation message (444) may be any indicator (e.g., a text object, a button) that conveys the newly selected target preset.

In FIG. 9C, after detecting an anatomical marker that indicates “Preset B” with a confidence level above a predetermined threshold, the user interface of the processing device (204) is modified by displaying a suggestion message (442) and a switch button (446) on the display screen (208). The switch button (446) is a control element that cause the “Preset A” in “Auto Mode” to switch to the determined target preset, “Preset B.” Interacting with the switch button (446) may be performed by touching a touchscreen interface of the display screen (208) or any method that the processing device (204) is configured to accept user input.

In FIG. 9D, after detecting an anatomical marker that indicates “Preset B” with a confidence level above a predetermined threshold, the processing device (204) displays a switch button (446) with a timer on the display screen (208). In one or more embodiments, the processing device (204) does not switch presets or switch to ultrasound image (110B) if the POCUS user does not activate the switch button (446) before the timer runs out. When the timer runs out, the switch button (446) may be removed from the display screen (208) and the processing device (204) remains in the “Preset A” in “Auto Mode,” as shown in FIG. 9D.

In FIG. 9E, after detecting an anatomical marker that indicates “Preset B” with a confidence level above a predetermined threshold, the processing device (204) switches from ultrasound image (110A) to ultrasound image (110B) and displays a switch button (446) with a timer on the display screen (208). In one or more embodiments, the processing device (204) does not switch from the “Preset A” in “Auto Mode” to “Preset B” but simply displays ultrasound image (100B) as a preview of “Preset B.” When the timer runs out, the switch button (446) may be removed from the display screen (208) and the processing device (204) reverts back to ultrasound image (110A) while remaining in the “Preset A” in “Auto Mode.”

In one or more embodiments, the processing device (204) does not switch presets or switch to ultrasound image (110B) until the POCUS user interacts with the processing device (204) to activate the switch button (446). In one or more embodiments, based on the POCUS user not activating the switch button (446), the processing device (204) may disable “Preset B” for a limited amount of time (e.g., to prevent repeating the suggestion when the POCUS user does not intend to use it).

The embodiments shown in FIGS. 9A-9E are non-limiting examples. Other display screen (208) configurations may be used without departing from the scope of the disclosure. For example, any combination of suggestion message (442), suggestion confirmation (444), switch button (446), update to the status interface (430), update to the control interface (410), update to the preset menu (420), or other update to the processing device (204) may be used. Accordingly, the scope of the invention should not be limited by the examples depicted in FIGS. 9A-9E.

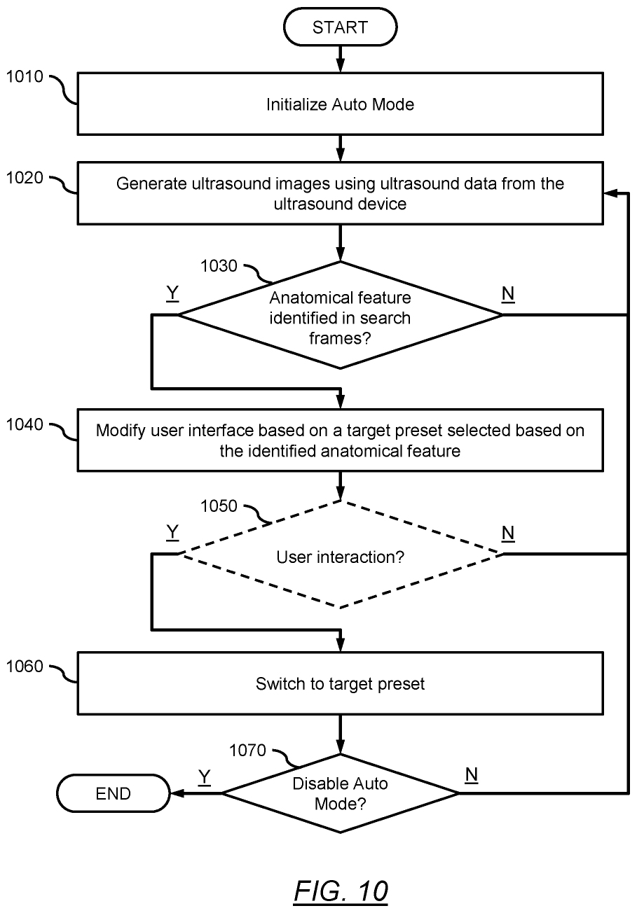

FIG. 10 shows a flowchart of a method in accordance with one or more embodiments. One or more of the individual processes in FIG. 10 may be performed by the processing device (204) of FIG. 2, as described above. One or more of the individual processes shown in FIG. 10 may be omitted, repeated, and/or performed in a different order than the order shown in FIG. 10. Accordingly, the scope of the invention should not be limited by the specific arrangement as depicted in FIG. 10.

At 1010, the “Auto Mode” is initialized on the processing device (204). For example, the “Auto Mode” may be a default mode of operation of the processing device (204) or a POCUS user may select the “Auto Mode” from a preset menu (420) to assist in collecting clinically relevant ultrasound images.

At 1020, the processing device (204) generates ultrasound images using ultrasound data from the ultrasound device (102). The ultrasound images include: a first portion of the ultrasound images that are imaging frames acquired with the first preset; and a second portion of the ultrasound images that are search frames acquired with a search preset.

As discussed above, the imaging frames are shown to the POCUS user on the display screen (208) of the processing device (204) and the search frames are used as inputs to a deep learning model (800) for identification of any anatomical feature during the scan.

At 1030, the processing device (204) determines whether or not an anatomical feature has been identified in the search frames by the deep learning model. In one or more embodiments, for the determination at 1030 to be YES, the anatomical feature must be identified with a confidence level that exceeds a predetermined threshold.

When the determination at 1030 is YES, the process continues to 1040.

When the determination at 1030 is NO, the process returns to 1020 to continue acquiring ultrasound data.

At 1040, the processing device (204) modifies a user interface based on a target preset selected based on the identified anatomical feature (e.g., a Lung preset is selected when the identified anatomical feature is a lung). As discussed above with respect to the non-limiting examples FIGS. 9A-9E, the display screen (208) may be modified by any combination of suggestion message (442), suggestion confirmation (444), switch button (446), update to the status interface (430), update to the control interface (410), update to the preset menu (420), or other update to the processing device (204).

At optional 1050, when the modification to the user interface includes a control element (e.g., switch button (446)), the processing device (204) determines whether or not the POCUS user interacts with the control element.

When the determination at 1050 is YES, the process continues to 1060. When the determination at 1050 is omitted or no control element is included in the modification to the user interface, the process may directly proceed from 1040 to 1060.

When the determination at 1050 is NO, the process returns to 1020 to continue acquiring ultrasound data.

At 1060, the processing device (204) switches to the target preset. Based on the target preset, the processing device (204) may update one or more modes used to control the ultrasound device (102) and one or more tools to analyze ultrasound data from the ultrasound device (102). For example, when switching to the new presets, all modes (e.g., Doppler, Biplane, Needle) and tools (e.g., Measurement, Labels) specific to the target preset become available.

At 1070, the processing device (204) determines whether or not “Auto Mode” is to be disabled. For example, the new target preset may provide the POCUS user with the optimal imaging preset for the current scan and the POCUS disables the “Auto Mode” to avoid further interruptions. In one or more embodiments, “Auto Mode” may be disabled automatically when the target preset is activated.

When the determination at 1070 is YES, the process ends.

When the determination at 1070 is NO, the process returns to 1020 to continue acquiring ultrasound data.

FIG. 11 shows a computing system (1100) in accordance with one or more embodiments. The computing system (1100) may be one or more mobile devices (e.g., laptop computer, smart phone, personal digital assistant, tablet computer, or other mobile device), desktop computers, servers, blades in a server chassis, or any other type of computing device or devices that includes at least the minimum processing power, memory, and input and output device(s) to perform one or more embodiments of the invention. As such, the computing system (1100) may be implemented as the processing device (204) described above with respect to FIG. 2.

As shown in FIG. 11, the computing system (1100) may include one or more processors (1105) (e.g., central processing unit (CPU), graphics processing unit (GPU), tensor processing unit (TPU), an integrated circuit for processing instructions, one or more cores or micro-cores of a processor) and one or more memory (1110) (e.g., random access memory (RAM), cache memory, flash memory, storage device, hard disk, an optical drive such as a compact disk (CD) drive or digital versatile disk (DVD) drive) for storing information.

The computing system (1100) may also include one or more input device(s) (1120), such as an ultrasound device, touchscreen, keyboard, mouse, microphone, touchpad, electronic pen, or any other type of input device.

Further, the computing system (1100) may include one or more output device(s) (1125), such as a screen (e.g., a liquid crystal display (LCD), a plasma display, touchscreen, cathode ray tube (CRT) monitor, projector, or other display device), a printer, external storage, or any other output device (e.g., speaker). One or more of the output device(s) may be the same or different from the input device(s).

The computing system (1100) may be connected to a network (1130) (e.g., a local area network (LAN), a wide area network (WAN) such as the Internet, mobile network, or any other type of network) via a network interface connection (not shown). The input and output device(s) may be locally or remotely (e.g., via the network (1130)) connected to the computer processor(s) (1105), memory (1110), and storage device(s) (1115). Many different types of computing systems exist, and the aforementioned input and output device(s) may take other forms.

Software instructions in the form of computer readable program code to perform embodiments of the invention may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that when executed by a processor(s), is configured to perform embodiments of the invention.

Further, one or more elements of the aforementioned computing system (1100) may be located at a remote location and be connected to the other elements over a network (1130). Further, one or more embodiments of the invention may be implemented on a distributed system having a plurality of nodes, where each portion of the invention may be located on a different node within the distributed system. In one embodiment of the invention, the node corresponds to a distinct computing device. Alternatively, the node may correspond to a computer processor with associated physical memory. The node may alternatively correspond to a computer processor or micro-core of a computer processor with shared memory and/or resources.

One or more of the embodiments of the invention may have one or more of the following improvements to performing ultrasound imaging: One or more embodiments aim to remove the step of selecting preset when scanning. This removes potential for error (e.g., a POCUS user scanning an organ in the wrong preset and getting suboptimal image and diagnosis), and focuses the clinician on image acquisition and interpretation as opposed to interacting with the device controls. One or more of the embodiments of the invention further demonstrate a practical application of improving ultrasound examinations. Furthermore, one or more of the embodiments of the invention further demonstrate performance of computer hardware systems.

In summary, POCUS users are often inexperienced and working under pressure. A successful POCUS user interface is simple and removes room for error and extra need for interaction with the device. Ultrasound imaging modes are optimized according to the expected anatomy to be encountered and exam type. For instance, when imaging superficial anatomy such as in the musculoskeletal application, the device will use a shallow field of view and high imaging frequencies. When imaging deeper anatomy such as in an abdominal exam, the device will use a large field of view and low imaging frequencies. When imaging fast moving structures such as the heart, the device will optimize for frame rate possibly at the expense of some image quality. When looking for specific ultrasound artefact signatures such as in lung imaging, the device will make little use of postprocessing in order to not remove these artefacts.

In one or more embodiments, matching the optimization to the anatomy/exam type can be done with presets. For example, when the processing device (204) has 22 different available presets, the machine is tuned differently for each exam type that can be encountered in clinical practice. In one or more embodiments, aim to remove the step of selecting preset when scanning. This removes potential for error (e.g., a POCUS user scanning an organ in the wrong preset and getting suboptimal image and diagnosis), and focuses the clinician on image acquisition and interpretation as opposed to interacting with the device controls.

In one or more embodiments, from an operational perspective, the workflow of the processing device (204) is as follows:
1. Boot up the “Auto-preset,”
2. Scan the body with a search preset that is able to cover a wide range of anatomies (e.g., close to today's Abdomen optimization”).
3. As the POCUS user scans the body, the processing device (204) is constantly identifying the anatomy being imaged.
4. When the processing device (204) detects an organ with high confidence, it either:
(i) switches imaging to the relevant preset (e.g., if a lung was identified, switch to the Lung preset): or
(ii) suggests to the user to switch presets, with (e.g., a popup message (“Lung detected: switch to lung?”)) or a popup message accompanied by an example image in the new preset (FIG. 9C).
5. When switching to the target presets, all modes (e.g., Doppler, Biplane, Needle, etc. . . . ) and tools (e.g., Measurement, Labels, etc. . . . ) specific to the identified preset become available.

In one or more embodiments, the basic implementation is as follows: In each preset, a “search frame” is added and time-interleaved with the actual imaging frames at a rate slow enough to not interfere with imaging (e.g., one search frame every 10 imaging frames). The search frame may be designed to be “one size fits all” (i.e., is able to image any anatomy decently). For example, it can be a mid-frequency (˜3.5 MHz), mid-depth (˜10 cm), mid field of view (“Curvilinear” geometry), fast (Nyquist-sampled, no compounding) sequence. Gain compensation may be applied to the search frames, but no image processing. Gain compensation is useful to narrow the input distribution of images. Avoiding image processing may be done to ensure stability over re-optimizations over the product life cycle.

In one or more embodiments, the image obtained from the “search frame” image is not show to the user, but the software uses the search frame for anatomy identification, by means of a neural network trained on data acquired with the search frame. Example imaging sequences are depicted in FIGS. 6A-7B, with search frames sparsely interleaved with frames from the currently selected preset. The time between the first search frame and the fourth search frame may be ˜1 second.

There are several advantages to using a search frame for anatomy identification. Training of the neural network classifier (810) only needs to happen on the data from the search frame (i.e., avoid the complexity of training a network for an anatomy recognition on data coming from different distributions (all image aspect ratios, resolutions, and appearance corresponding to all presets)). In addition, re-training is not required because, while the imaging modes get updated and re-optimized over time, the search frame can be maintained with little or no changes over time.

In this case, a neural network classifier (810) would have 1 input (the image from the search frame) and as many outputs as available presets (i.e., labels corresponding to the presets). Excluding data augmentation, training would involve acquiring all clinically relevant anatomies with this search frame.

### Alternative Embodiments

In one or more embodiments, the search frame strategy is to detect the anatomy being imaged on whatever preset is in current use. It can be a viable strategy if the total number of presets to image with is small. For instance, one could imagine a simplified software where only four presets are in use, (e.g., Cardiac, Abdominal, Lung, and Superficial). However, acquiring relevant training data and maintaining neural network performance over preset re-optimizations is more complicated than in the “search frame” approach.

In this case, a neural network classifier (810) would have 1 input (the image from the preset in current use) and as many outputs as available presets (i.e., labels corresponding to the presets)). Training may be hard due to the difficulty and effort in acquiring relevant data (all anatomies of interest need to be imaged in all presets of interest).

In one or more embodiments, a small number of “pillar” search frames are used.

To cater to the wide variety found in the available presets, four different pillar presets are used (e.g., Abdominal, Cardiac, Lung, and Musculoskeletal. The assumption is that each of these presets should be able to image reasonably well anatomies seen in a variety of different presets, for instance:
Cardiac covers Cardiac Standard, Cardiac Deep, Coherence, Pediatric Cardiac, etc.
Abdomen covers Abdomen, Bladder, Fast, Aorta/gallbladder, Pediatric abdomen, etc.
MSK covers MSK, Vascular access, Carotid, Small parts, Thyroid, etc.,
Lung covers Lung, pediatric lung, etc.

An example imaging sequence is depicted in FIGS. 6B and 7B, with search frames from a limited amount of pillar presets sparsely interleaved with frames from the currently selected preset. The time between the first search frame and the fourth search frame may be ˜1 second.

In one or more embodiments, a neural network classifier (810) would have 4 inputs (i.e., the images obtained by all of the four search frames) and up to 22 outputs (i.e., labels corresponding to the presets). Training would ideally involve acquiring all anatomies of interest in these four presets.

There are two advantages of using a small number of “pillar” presets that closely resemble some of our released presets, instead of a single “one size fits all” search frame:

The first advantage is the availability of data: large amounts of data acquired on the cloud by many users, and annotate them manually or automatically in order to pre-train a neural network for anatomy identification.

The second advantage is that since the anatomy being scanned is looked at in four different ways with different parameters, it should bring robustness to the classification, as each different search frames bring different features that the artificial intelligence (AI) can leverage.

This embodiment thus allows potentially better classification than single search frame, while keeping complexity in check.

In one or more embodiments, as an alternative to using four presets close to some released presets as search frames, one could use four custom presets designed to sample the imaging parameter space evenly. This is an extension of the “one size fits all” search frame. For instance, these four search frames could be mostly based on depth (e.g., 3 cm, 6 cm, 12 cm, and 18 cm depth, with transmit frequency and lateral beam spread scaling correspondingly). In general, the first preset may be high frequency, shallow depth, the last preset may be low frequency, deep depth, and the other two may be in between. A rule of thumb for lateral beam spacing in a sector scan is that the transmit beams angular spacing have to be equal to lambda/A where A is the size of the active aperture and lambda the wavelength (inversely proportional to frequency). The imaging lines are spaced twice to three times tighter. For total angular spread, 0 degree (rectangular scan) may be used for the shallow preset (looks like the MSK preset), and +/−45 degrees for the deeper preset (looks like the Abdominal preset), and the other 2 presets are in between

In one or more embodiments, one could imagine adding one fifth “search frame” to capture temporal dynamics of the anatomy of interest (e.g., to identify heart). An M-mode line can be added and its evolution over time used as a fifth “search frame.” Motion could not be captured in the other frames for practical limitations (neural network size explosion when looking at 3D data) and simply because the search frames may be interleaved at a rate that is too slow (interfere with imaging). By default, the M-mode line may be placed down the middle. The rate of the M-mode line is higher than that of the search frames so temporal dynamics can be captured. The other search frames are triggered at a rate around ˜1 Hz, and fed frame by frame to the neural network. A frame in these normal search frames may be the intensity distribution over two spatial dimensions (azimuth and depth) at one time, (i.e., I(x,y, t=t0)). For M-mode, the intensity distribution over time (say, one second) and depth at one azimuth, (i.e., I(x=x0, y, t)) may be used. Ideally the fifth frame is sent to the neural network at the same rate as the other search frames. See FIG. 7A.

In one or more embodiments, in order to minimize disruption to imaging, the search frames need not be activated in the background at all times. For example, triggering can occur when an image quality indicator (e.g., determined as described in US20200372657A1 and/or U.S. Pat. No. 10,628,932) drops below a certain value for a certain amount of frames. The search frames can ideally be triggered from any preset, not just from one of the 4 pillar presets.

Although the disclosure has been described with respect to a limited number of embodiments, those skilled in the art, having benefit of this disclosure, will appreciate that various other embodiments may be devised without departing from the scope of the present invention. Accordingly, the scope of the invention should be limited only by the attached claims.

### REFERENCE NUMERALS

100 ultrasound system
101 subject
102 ultrasound device
110 ultrasound image
112 communication link
152 transducer array
154 transmitter (TX) circuitry
156 receiver (RX) circuitry
158 timing and control circuitry
160 signal conditioning/processing circuitry
162 semiconductor die
164 serial output signal
166 clock signal
168 power management circuitry
170 high intensity focused ultrasound (HIFU) circuitry
Vin voltage supply
GND ground line
204 processing device
208 display screen
210 processor
212 memory
214 input device
216 camera
220 network
230 server
300 phase array
Eᵢ transducer
Φᵢ phase
τᵢ time delay
301ᵢ signal driver
302 acoustic beam
304 angled acoustic beam
410 control interface
420 preset menu
422 auto mode button
424i predetermined preset button
426 selection button
430 status interface
442 suggestion message
444 suggestion confirmation
446 switch button

What is claimed is:

1. A processing device that communicates with an ultrasound device, the processing device comprising:
a display screen;
a memory that stores presets, where each preset includes one or more modes used to control the ultrasound device and one or more tools to analyze ultrasound data from the ultrasound device; and
a processor coupled to the memory,
wherein the processor is configured to:
operate the ultrasound device using a first preset;
generate ultrasound images using ultrasound data from the ultrasound device, where the ultrasound images include:
a first portion of the ultrasound images that are imaging frames acquired with the first preset; and
a second portion of the ultrasound images that are search frames acquired with a search preset;
display the imaging frames of the first portion on the display screen;
identify an anatomical feature in the search frames using a deep learning model;
select a target preset based on the identified anatomical feature; and
modify a user interface of the processing device based on the target preset,
wherein the search frames are time-interleaved with the imaging frames.

2. The processing device of claim 1,
wherein the search preset is different from the first preset,
wherein the search frames include two-dimensional ultrasound images for the search preset,
wherein the search preset utilizes a Nyquist sampling rate and no image processing such that the second portion of ultrasound images are generated faster than the first portion of images,
wherein the deep learning model is a neural network classifier trained to identify the anatomical feature based on image recognition,
wherein the neural network classifier is trained with ultrasound images generated using parameters of the search preset.

3. The processing device of claim 1,
wherein the search preset is the same as the first preset,
wherein the search frames and the imaging frames are two-dimensional ultrasound images generated using the first preset,
wherein the deep learning model is a neural network classifier trained to identify the anatomical feature based on image recognition,
wherein the neural network classifier is trained with ultrasound images generated using parameters of each of the presets stored in the memory.

4. The processing device of claim 1,
wherein the search preset includes a plurality of different pillar presets that are different from the first preset,
wherein the search frames include two-dimensional ultrasound images for each of the different pillar presets,
wherein the deep learning model is a neural network classifier trained to identify the anatomical feature based on image recognition,
wherein the neural network classifier is trained with ultrasound images generated using parameters of each of the different pillar presets.

5. The processing device of claim 4,
wherein each of the plurality of pillar presets utilizes a different combination of frequency and imaging depth parameters.

6. The processing device of claim 4,
wherein the plurality of different pillar presets include four pillar presets corresponding to a cardiac anatomical region, an abdominal anatomical region, a musculoskeletal anatomical region, and a lung region.

7. The processing device of claim 1,
wherein the search preset includes a line scan mode that is different from the first preset,
wherein the search frames include one-dimensional line scan images that are time-interleaved between every imaging frame,
wherein the deep learning model is a neural network classifier trained to identify the anatomical feature based on temporal dynamics in the line scan images,
wherein the neural network classifier is trained with time-varying line scan images generated using parameters of the line scan mode.

8. The processing device of claim 7,
wherein the search preset further includes a plurality of different pillar presets that are different from the first preset,
wherein the search frames include two-dimensional ultrasound images for each of the different pillar presets,
wherein the neural network classifier is further trained to identify the anatomical feature based on image recognition of the two-dimensional ultrasound images,
wherein the neural network classifier is further trained with ultrasound images generated using parameters of each of the different pillar presets.

9. The processing device of claim 1,
wherein the processor is further configured to determine an image quality of the search frames during analysis for the anatomical feature,
wherein, in response to the image quality being greater than or equal to a predetermined threshold, the search frames are analyzed for the anatomical feature,
wherein, in response to the image quality being less than the predetermined threshold, the search frames are deleted from the processing device.

10. The processing device of claim 1,
wherein modifying the user interface includes automatically switching from the first preset to the target preset without interaction from a user of the processing device.

11. The processing device of claim 10,
wherein modifying the user interface further includes indicating the anatomical feature has been identified on the display screen before automatically switching from the first preset to the target preset.

12. The processing device of claim 1,
wherein modifying the user interface includes creating a control element for the target preset on the display screen,
wherein the first preset is switched to the target preset in response to a user of the processing device interacting with the control element.

13. The processing device of claim 12,
wherein modifying the user interface includes:
creating a first timer that limits an amount of time available for the user to interact with the control element; and
creating a second timer that disables the target preset from selection based on the identified anatomical feature for a predetermined amount of time.

14. A method of operating a processing device that communicates with an ultrasound device, the method comprising:
operating the ultrasound device using a first preset of a plurality of presets stored in a memory of the processing device, where each preset includes one or more modes used to control the ultrasound device and one or more tools to analyze ultrasound data from the ultrasound device;
generating ultrasound images using ultrasound data from the ultrasound device, where the ultrasound images include:
a first portion of the ultrasound images that are imaging frames acquired with the first preset; and
a second portion of the ultrasound images that are search frames acquired with a search preset;
displaying the imaging frames on a display screen of the processing device;
identifying an anatomical feature in the search frames using a deep learning model;
selecting, in response to identifying the anatomical feature, a target preset based on the identified anatomical feature; and
modifying a user interface of the processing device based on the target preset,
wherein the search frames are time-interleaved with the imaging frames.

15. A non-transitory computer readable medium (CRM) storing computer readable program code for operating a processing device that communicates with an ultrasound device, the computer readable program code causes the processing device to:
operate the ultrasound device using a first preset of a plurality of presets stored in a memory of the processing device, where each preset includes one or more modes used to control the ultrasound device and one or more tools to analyze ultrasound data from the ultrasound device;
generate ultrasound images using ultrasound data from the ultrasound device, where the ultrasound images include:
a first portion of the ultrasound images that are imaging frames acquired with the first preset; and
a second portion of the ultrasound images that are search frames acquired with a search preset;
display the imaging frames on a display screen of the processing device;
identify an anatomical feature in the search frames using a deep learning model;
select, in response to identifying the anatomical feature, a target preset based on the identified anatomical feature; and
modify a user interface of the processing device based on the target preset,
wherein the search frames are time-interleaved with the imaging frames.
